(19) United States
(12) Patent Application Publication
(10) Pub. No.: US 2001/0046968 A1
(43) Pub. Date: Nov. 29, 2001

(54) Opioid growth factor modulates angiogenesis

(76) Inventors: Ian S. Zagon, Hummelstown, PA (US); John Blebea, Hummelstown, PA (US); Patricia J. McLaughlin, Harrisburg, PA (US)

Correspondence Address: Leopold Presser, Scully, Scott, Murphy & Presser, 400 Garden City Plaza, Garden City, NY, 11530, US

(21) Appl. No.: 09/815,069

(22) Filed: Mar. 22, 2001

Related U.S. Application Data
(60) Provisional application No. 60/191,522, filed on Mar. 23, 2000.

(51) Int. Cl. A61K 48/00; A61K 39/395; A61K 31/485

(52) U.S. Cl. 514/44; 514/282; 424/139.1

(57) ABSTRACT

The present invention provides that an endogenous opioid peptide, OGF, inhibits angiogenesis in vivo via acting on OGF receptor. The present inventions also provides that opioid antagonist naltrexone and naltrexone stimulated blood vessel development. Therapeutic compositions and methods for modulating angiogenesis are provided.

### CROSS REFERENCE TO RELATED APPLICATION

This application claims the benefit of U.S. Provisional Application No. 60/191,522, filed on Mar. 23, 2000.

### FIELD OF THE INVENTION

The present invention relates to the use of opioid growth factor (“OGF”) and OGF antagonists in the modulation of angiogenesis.

### BACKGROUND OF THE INVENTION

Atherosclerosis affects more than 20 million people in the United States (Thorn T. J. et al., Hurst's The Heart, Arteries, and Veins, 9^{th} Ed. McGraw-Hill; 3-17 (1998)). Approximately one million patients undergo either coronary artery bypass or balloon angioplasty/stenting each year, while more than 100,000 individuals have lower extremity arterial revascularization (Stanley J. C. et al., J. Vasc. Surg., 23: 172-181 (1996)). Additionally, more than 100,000 people require leg amputations each year because of infection or advanced atherosclerotic disease not amenable to present revascularization modalities. Recent studies have suggested that exogenous angiogenic growth factors can stimulate the peripheral development of collateral arteries in patients with critical limb ischemia, thus inducing therapeutic angiogenesis and avoiding amputations in selected patients (Baumgartner I. et al., Circulation, 97: 1114-1123 (1998); Isner J. M. et al., Lancet, 348: 370-347 (1996); Tsurumi Y. et al., Circulation, 96: II382-11388 (1997)). Conversely, inhibition of angiogenesis has been studied in patients with life threatening hemangiomas, ocular neovascularization, and cancer (Folkman J., NEJM, 333: 1757-1763 (1995); Folkman J. et al., J. Biol,. Chem., 16: 10931-10934 (1992)).

The pentapeptide [Met⁵]-enkephalin is an endogenous opioid directly involved in growth processes and serves as a negative regulator in a wide variety of cells and tissues (Zagon I. S. et al., Neuroscience, 37: 223-226 (1990); Zagon I. S. et al., Brain Res., 551: 181-186 (1990)). In view of the function and distribution of [Met⁵]-enkephalin, this peptide has been termed “opioid growth factor” (OGF). The effects of OGF are dose-related, non-cytoxic, receptor-mediated, and effective at concentrations consistent with the binding affinity of OGF receptor. OGF also has been shown to be an autocrine produced peptide that inhibits DNA synthesis in endothelial, smooth muscle cells, and fibroblasts in the rat aorta (Zagon I. S. et al., Am. J Physiol, 270: R22-R32 (1996); Wu Y. et al., Dev. Dynam, 211: 327-337 (1998)).

The present invention demonstrates for the first time that endogenous opioids modulated angiogenesis in vivo. Accordingly, the present invention provides novel methods and pharmaceutical compositions for modulating angiogenesis.

### SUMMARY OF THE INVENTION

One embodiment of the present invention provides pharmaceutical compositions capable of modulating angiogenesis.

A preferred pharmaceutical composition of the present invention includes a therapeutically effective amount of opioid growth factor (or “OGF”) sufficient to inhibit angiogenesis in a subject.

Another preferred pharmaceutical composition of the present invention includes a therapeutically effective amount of naltrexone sufficient to cause intermittent blockade of OGF receptors and therefore inhibit angiogenesis.

Still another pharmaceutical composition of the present invention includes a therapeutically effective amount of a sequence coding for preproenkephalin or a sequence coding for OGF receptor.

Another preferred pharmaceutical composition of the present invention includes a therapeutically effective amount of an OGF antagonist which enhances angiogenesis. Preferred OGF antagonists for use in the pharmaceutical compositions of the present invention include both short-acting opioid antagonists (e.g., naloxone) and long-acting opioid antagonists (e.g., naltrexone), as well as antibodies against OGF or OGF receptors, antisense molecules of the gene encoding for preproenkephalin and antisense molecules of the OGF-receptor-encoding gene.

OGF or an OGF antagonist is preferably provided in a pharmaceutically acceptable carrier, formulated in any form suitable for administration to a subject via injection or implantation.

In a further aspect of the invention, the pharmaceutical compositions of the present invention are used to modulate angiogenesis in a subject.

In one embodiment, the present invention provides methods of inhibiting angiogenesis in a subject in need thereof by administering to the subject a therapeutically effective amount of OGF, or an amount of naltrexone necessary to cause an intermittent blockade of OGF receptors.

A subject in need of inhibition of angiogenesis includes patients suffering hemangiomas, ocular neovascularization, tumors, metastatic tumors, venous malformations, varicose veins, arterio-venous malformations, vascular tumors, intimal hyperplasia (following angioplasty, stenting, stented grafts, bypasses, or other endovascular or extra-vascular interventions), and/or atherosclerotic arterial stenosis or occlusions.

In another embodiment, the present invention provides methods of enhancing angiogenesis in a subject in need thereof by administering to the subject a therapeutically effective amount of an OGF antagonist.

Preferred OGF antagonists for use in the pharmaceutical compositions of the present invention include both short-acting opioid antagonists (e.g., naloxone) and long-acting opioid antagonists (e.g., naltrexone), as well as antibodies against OGF or OGF receptors and antisense molecules of the OGF-receptor-encoding gene.

A subject in need of enhanced angiogenesis includes patients suffering from arterial insufficiency in the upper or lower extremities, peripheral venous insufficiency, deep venous thrombosis, organ arterial insufficiency (e.g., brain, heart, liver, kidneys, muscle), and lymphedema.

The pharmaceutical compositions of the present invention can be administered to the subject in need thereof by topical, parenteral, or intravenous/intraarterial injection, oral ingestion, or by implantation near or in a pre-selected tissue or organ. Other methods of delivery include time-released mechanisms that include a patch, microcapsule, microperfusion, infuson design catheter, an implant, a suspension or an osmotic pump.

### BRIEF DESCRIPTION OF THE DRAWINGS

FIGS. 1A-1D. Digitized computer images of seven-day old CAMs. In contrast to control specimens (A), CAMs treated with retinoic acid (B) exhibited a marked decrease in blood vessel growth in the 100 mm² region around the disk. Following treatment with OGF (C), blood vessel growth was suppressed compared to control samples. Vessel inhibition is no longer seen following treatment with naltrexone (D). Note the circle representing the applied disk (with cross-hatched arrow) and surrounding blood vessels (arrow). Bar=5 mm.

FIG. 2. Comparison of the total number of blood vessels in the region around the applied disks. The number of blood vessels were significantly decreases in the OGF group and increased in the NTX group compared to Control preparations. Significant difference from controls:*p<0.005, ^{#}p<0.001.

FIG. 3. Total blood vessel length was significantly inhibited in CAMs exposed to OGF and increased following treatment with NTX. Simultaneous treatment with NAL prevented inhibition by OGF. Significant difference from controls:*p<0.005.

FIG. 4. Mean blood vessel length was significantly increases by treatment with OGF while NTX decreased mean blood vessel length. Significant differences form controls: ^{#}p<0.001, ⁺p<0.0005.

FIGS. 5A-5E. Immunocytochemical preparation of isolated principal CAM vessels. Cross-section of a vessel stained with hematoxylin-eosin demonstrating its morphologic structure at this early developmental stage with flattened inner endothelial cells (arrows) and messenchymal presumptive immature smooth muscle cells (curved arrow) (A). Sections of vessels incubated with antibodies to OGF (B, C) or the OGF receptor (D, E) at 1:100 dilutions. Note staining of cytoplasm (arrowheads) of cells throughout the wall of the vessels and non-staining of the nuclei. C and E are control sections stained with antibodies to OGF and OGF receptor, respectively, following pre-absorption with excess OGF and OGF receptor. They did not exhibit any immunofluorescence. Times of exposure and printing correspond in all photos. Bar=48 μm for A and 16 μm for B-E.

FIG. 6. In another series of experiments, the angiogenic inhibitor RA significantly diminished the number of blood vessels while VEGF markedly increased angiogenesis (§p<0.0001).

FIG. 7. RA decreased the total length of measured blood vessels while VEGF induced a marked increase as compared to controls (§p<0.0001).

FIG. 8. Inhibition of new vessel development by RA increase the calculated mean vessel length while VEGF was associated with decreased values compared to control preparations (§p<0.0001).

### DETAILED DESCRIPTION OF THE INVENTION

The present inventors have demonstrated for the first time that an endogenous opioid peptide, OGF, inhibits angiogenesis in vivo. The present inventors have also demonstrated that the effect of OGF on angiogenesis is mediated through its receptor, OGFr, because naloxone, the opioid receptor antagonist, blocked OGF's suppressive activities on blood vessels growth when administered concomitantly with OGF. In addition, the present invention has demonstrated that the potent and longer-acting opioid antagonist naltrexone, through a persistent blockade of opioid-receptor interaction, markedly stimulated blood vessel development. Accordingly, the present invention provides therapeutic compositions and methods for modulating angiogenesis in a subject in need thereof.

As used herein, the term “angiogenesis” refers to the process by which new blood vessels, including arteries and veins, are formed into a tissue or organ or portion of the body with accompanying blood circulation. The process of angiogenesis involves migration and proliferation of endothelial cells which line the lumen of blood vessels, smooth muscle cells, extracellular matrix components, and associated growth factors.

The term “modulating” as used herein encompasses both enhancing and inhibiting. By “enhancing angiogenesis” is meant stimulating, accelerating, or potentiating the process of blood vessel formation of large and small vessels (including arteries, veins, and lymphatic vessels), as well as capillaries. The terms “enhancing angiogenesis” and “increased vascularization” are used herein interchangeably. By “inhibiting angiogenesis” is meant preventing or reducing the formation of blood vessels including arteries, veins, lymphatics, and capillaries.

The term “subject” is taken to mean any animal subject, preferably, a human subject. Inhibition of angiogenesis is desired in a subject suffering hemangiomas, ocular neovascularization, tumors, metastatic tumors, venous malformations, varicose veins, arterio-venous malformations, vascular tumors, intimal hyperplasia (following angioplasty, stenting, stented grafts, bypasses, or other endovascular or extra-vascular interventions), and/or atherosclerotic arterial stenosis or occlusions. Enhanced angiogenesis is desired in a subject suffering a condition where vascularization is inadequate and angiogenesis is clinically required, for example, in the treatment of arterial insufficiency in the upper or lower extremities, peripheral venous insufficiency, deep venous thrombosis, organ arterial insufficiency (e.g., brain, heart, liver, kidneys, muscle), and lymphedema.

One aspect of the present invention is directed to pharmaceutical compositions capable of modulating angiogenesis.

In one embodiment, the present invention provides pharmaceutical compositions which inhibit angiogenesis.

A preferred angiogenesis-inhibiting composition of the present invention includes a therapeutically effective amount of opioid growth factor (or “OGF”). The amount of OGF that is therapeutically effective refers to the amount of OGF sufficient to occupy OGF-specific receptors of the endothelial cells and the mesenchymal cells of the developing vessels, and cause inhibition of angiogenesis in a subject in need of such inhibition, such that the symptoms associated with the overabundant angiogenesis are prevented, delayed or eliminated, or the severity of such symptoms is reduced. The precise amount of OGF to be therapeutically effective depends upon the condition and weight of the subject, as well as the route of administration. As a general rule, for intravenous administration, regimes in cumulative amounts ranging from about 10 mg to about 30 mg (or about 25 μg/kg to about 300 μg/kg) exogenous OGF per day for a human patient is effective. For subcutaneous administration, regimes in cumulative amounts ranging from about 1 mg/ml to about 5 mg/ml (or about 10 μg/kg to about 50 μg/kg) of exogenous OGF per day are effective. For administration via a minipump, OGF can be administered at about 10⁻⁶ M.

Another preferred angiogenesis-inhibiting composition of the present invention includes a therapeutically effective amount of naltrexone which causes an intermittent or temporary blockade of OGF receptors. An intermittent or temporary blockade of OGF receptors induces an elevated level of endogenous OGF and/or elevated numbers of OGF receptors. Thus, angiogenesis can be inhibited by the interaction of induced increased levels of endogenous OGF and receptor. The precise amount of naltrexone to be therapeutically effective in inhibiting angiogenesis depends upon the condition and weight of the subject, as well as the route of administration. In general, regimes ranging in cumulative amounts from about 1 to about 5 mg of naltrexone per day for a human patient can effect successful inhibition of angiogenesis.

Another angiogenesis-inhibiting pharmaceutical composition of the present invention includes a therapeutically effective amount of a sequence coding for preproenkephalin or a sequence coding for the OGF receptor.

The angiogenesis-inhibiting compositions of the present invention can include other substances that are appropriate or beneficial for inhibiting angiogenesis, for example, angiostatin as described by Cramer, D. A. Annals Intern Med 129:841-843, 1998; Griffloen, A. W. and Molema, G. Pharmacol. Rev. 52:237-268, 2000, incorporated herein by reference.

In another embodiment, the present invention provides pharmaceutical compositions which enhance angiogenesis.

A preferred angiogenesis-enhancing composition of the present invention includes a therapeutically effective amount of at least one OGF antagonist.

Preferred OGF antagonists to be used in the angiogenesis-enhancing compositions of the present invention include short-acting and long-acting opioid antagonists, e.g., naloxone or naltrexone. Other OGF antagonists which can be used in the angiogenesis-enhancing compositions of the present invention include antibodies specific for OGF, antibodies specific for OGF receptors, as well as antisense molecules of the preproenkephalin-encoding gene and antisense molecules of the OGF receptor-encoding gene. The angiogenesis-enhancing compositions of the present invention can include one or more of the foregoing OGF antagonists.

Without intending to be limited to any particular theory, it is believed that persistent blockade of OGF receptors or a persistent sequestering of endogenous OGF by an OGF antagonist reduces the synthesis of endogenous OGF through a feedback system. As the inhibitory effect of OGF on angiogenesis appears to require constitutive expression of OGF, the reduction of OGF synthesis caused by an OGF antagonist therefore leads to an increased formation of blood vessels.

The amount of the OGF antagonist that is therapeutically effective refers to the amount of the OGF antagonist sufficient to cause a persistent blockade of OGF receptors or a persistent sequestering of endogenous OGF thereby enhancing angiogenesis in a subject in need of such enhancement, such that the symptoms associated with the inadequate angiogenesis are prevented, delayed or eliminated, or the severity of such symptoms is reduced. The precise amount of an OGF antagonist to be therapeutically effective can be determined by a physician based on the condition and weight of the subject, the nature of the antagonist and the route of administration. In general, the amount of naloxone necessary to cause a persistent blockade of the receptors is in the range of about 0.1 to about 15 mg per dose for a human patient, and preferably, about 1 mg to about 5 mg per dose, administered multiple times daily (preferably one dose every six hours). The amount of naltrexone necessary to cause a persistent blockade of the receptors is, in general, in the range of about 10 mg to about 30 mg per day. If a minipump is used for administration, an OGF antagonist can be administered at a concentration of 10⁻⁶ M for an appropriate period of time.

The angiogenesis-enhancing compositions of the present invention can include other substances that are appropriate or beneficial for angiogenesis. These substances can include any other angiogenic compounds, growth hormones, growth factors, biologically active segments of growth factors, interleukins, polysaccharides, or mixtures thereof. Specific examples include, but are not limited to, vascular endothelial growth factor (VEGF), pituitary growth hormones (hGH and bGH), various growth factors such as fibroblast growth factors (FGF), insulin-like growth factors (IGF), platelet-derived growth factors (PDGF), transforming growth factors (e.g., transforming growth factor alpha and beta), and other angiogenic compounds such as synthetic peptides Gly-His-Lys (GHK), Gly-Arg-Gly-Asp (GRGD) and Arg-Gly-Asp (RGD). See, e.g., U.S. Pat. Nos. 5,763,399 and 4,888,324, both of which are incorporated herein by reference.

In accordance with the present invention, OGF or an OGF antagonist of the pharmaceutical compositions of the present invention is preferably provided in a pharmaceutically acceptable carrier. The carrier can be liquid, semi-solid, e.g. pastes, or solid carriers. Except insofar as any conventional media, agent, diluent or carrier is detrimental to the recipient or to the therapeutic effectiveness of angiogenic substances or angiogenesis-inhibiting substances contained therein, its use in the pharmaceutical compositions of the present invention is appropriate. Examples of carriers include oils, water, saline solutions, gel, lipids, liposomes, resins, porous matrices, binders, fillers and the like, or combinations thereof.

Preferably, the carrier for use in the pharmaceutical compositions of the present invention is a controlled release matrix, a material which allows the slow release of OGF or an OGF antagonist mixed or admixed therein. Examples of such controlled release matrix material include, but are not limited to, sustained release biodegradable formulations described in U.S. Pat. No. 4,849,141 to Fujioka et al., U.S. Pat. No. 4,774,091 to Yamashira, U.S. Pat. No. 4,703,108 to Silver et al., and Brem et al. (J. Neurosurg. 74: 441-446, 1991), all of which are incorporated herein by reference.

In accordance with the present invention, OGF or an OGF antagonist and any other substances appropriate for use in the present pharmaceutical composition, can be combined with a pharmaceutical carrier in any convenient and practical manner, e.g., by admixture, solution, suspension, emulsification, encapsulation, absorption and the like, and can be made in formulations suitable for injections, implantations, inhalations, ingestions and the like. Preferable, the pharmaceutical compositions of the present invention are formulated in implantable pellet forms or in forms suitable for administration through controlled-release minipumps.

In a further aspect of the invention, a pharmaceutical composition as described hereinabove is administered to a subject to modulate angiogenesis.

In one embodiment, the present invention provides methods of inhibiting angiogenesis in a subject in need thereof by administering to the subject a therapeutically effective amount of OGF, preferably, with a pharmaceutically acceptable carrier.

A subject in need of inhibition of angiogenesis includes patients suffering hemangiomas, ocular neovascularization, tumors, metastatic tumors, venous malformations, varicose veins, arterio-venous malformations, vascular tumors, intimal hyperplasia (following angioplasty, stenting, stented grafts, bypasses, or other endovascular or extra-vascular interventions), and/or atherosclerotic arterial stenosis or occlusions.

The precise amount of OGF to be therapeutically effective (i.e., sufficient to occupy OGF receptors thereby inhibiting angiogenesis) depends upon the condition and weight of the subject, as well as the route of administration. As a general rule, for intravenous administration, regimes in cumulative amounts ranging from about 10 mg to about 30 mg (or about 25 μg/kg to about 300 μg/kg) exogenous OGF per day for a human patient is effective. For subcutaneous administration, regimes in cumulative amounts ranging from about 1 mg/ml to about 5 mg/ml (or about 10 μg/kg to about 50 μg/kg) of exogenous OGF per day are effective. For administration via a minipump, OGF can be administered at about 10⁻⁶ M. OGF can be administered in a single dose or in multiple doses, given either simultaneously or over an extended period of time.

In another embodiment, the present invention provides methods of inhibiting angiogenesis in a subject in need thereof by administering to the subject a therapeutically effective amount of naltrexone necessary to cause intermittent blockade of OGF receptors with a pharmaceutically acceptable carrier. The precise amount of naltrexone to be therapeutically effective in inhibiting angiogenesis depends upon the condition and weight of the subject, as well as the route of administration. In general, regimes ranging in cumulative amounts from about 1 to about 5 mg of naltrexone per day for a human patient can effect successful inhibition of angiogenesis.

In another embodiment, the present invention provides methods of inhibiting angiogenesis in a subject in need thereof by administering to the subject a therapeutically effective amount of a sequence coding for preproenkephalin or a sequence coding for OGF receptor.

In still another embodiment, the present invention provides methods of enhancing angiogenesis in a subject in need thereof by administering to the subject a therapeutically effective amount of an OGF antagonist with a pharmaceutically acceptable carrier.

Preferred OGF antagonists for administration include opioid antagonists, e.g., naloxone or naltrexone, antibodies specific for OGF, antibodies specific for OGF receptors, as well as antisense molecules of the preproenkephalin-encoding gene and antisense molecules of the OGF receptor-encoding gene. One or more of the foregoing OGF antagonists can be included in the administration.

A subject in need of enhanced angiogenesis includes patients suffering from arterial insufficiency in the upper or lower extremities, peripheral venous insufficiency, deep venous thrombosis, organ arterial insufficiency (e.g., brain, heart, liver, kidneys, muscle), and lymphedema.

The amount of an OGF antagonist to be therapeutically effective (i.e., sufficient to cause a persistent blockade of OGF receptors or a persistent sequestering of endogenous OGF, thereby enhancing angiogenesis) depends upon the condition and weight of the subject as well as the route of administration. In general, the amount of naloxone necessary to cause a persistent blockade of the receptors is in the range of about 0.1 to about 15 mg per dose for a human patient, and preferably, about 1 mg to about 5 mg per dose, administered multiple times daily (preferably one dose every six hours). The amount of naltrexone necessary to cause a persistent blockade of the receptors is, in general, in the range of about 10 mg to about 30 mg per day. If a minipump is used for administration, an OGF antagonist can be administered at a concentration of 10⁻⁶ M for an appropriate period of time.

An OGF antagonist can be administered in a single dose or in multiple doses, given either simultaneously or over an extended period of time. The amount of an OGF antagonist administered can be controlled such that angiogenesis is enhanced or accelerated in a tissue or organ to a desired extent, and then standard chemotherapeutic agents (e.g., cis platin) can be administered to the tissue or organ which kill dividing cells.

The pharmaceutical compositions of the present invention can be administered to the subject by standard routes, including the oral, ophthalmic, nasal, topical, transdermal, parenteral (e.g., intravenous, intraperitoneal, intradermal, subcutaneous or intramuscular) intracranial, intracerebral, intraspinal, intravaginal, intrauterine, or rectal route. Preferably, the compositions can also be introduced into the body, by injection or by surgical implantation or attachment, proximate to a preselected tissue or organ site such that a significant amount of an angiogenic substance or an angiogenesis-inhibiting substance is able to enter the site, preferably, in a controlled release fashion, by direct diffusion to induce the vascularization into the site.

All the publications mentioned in the present disclosure are incorporated herein by reference. The terms and expressions which have been employed in the present disclosure are used as terms of description and not of limitation, and there is no intention in the use of such terms and expressions of excluding any equivalents of the features shown and described or portions thereof, it being recognized that various modifications are possible within the scope of the invention.

The present invention is further illustrated, but is not limited, by the following examples.

### EXAMPLE 1

### MATERIALS AND METHODS

Chorioallantoic Model

The CAM model utilized in these studies has been described by Mazo et al. (FASEB J. 13: A527, 1999). In brief, fertilized white broiler chicken eggs were obtained from a local hatchery and kept at room temperature for 48 hours. The outside of the shells were washed with soap, and placed in a 70% ethyl alcohol bath for 30 seconds. The eggs were incubated at 37° C. for three days and rotated 180 degrees three times daily. After incubation, the eggs were cleansed with an iodine solution and dried on a sterile tray. The eggs were cracked, and the embryos explanted into spherical plastic 100 mm×25 mm weigh boats. Specimens were covered with a plastic lid, placed in a humidified chamber at 37° C., and maintained for 48 to 72 hours until the CAM achieved at least a 10 mm diameter.

Drug Delivery

Methylcellulose disks were prepared as reported by Mazo et al. (FASEB J. 13: A527, 1999). Five micrograms each of OGF (n=10), OGF and naloxone (NAL) simultaneously (n=9), NAL alone (n=11), or naltrexone (NTX)(n=10), were prepared in 10 μL of distilled water and combined in equal parts with a methylcellulose solution. These doses were selected on the basis of prior animal and culture studies (Zagon I. S. et al., Am. J Physiol, 270: R22-R32 (1996); McLaughlin P. J. et al., Int. J. Oncol., 14: 991-998 (1999)). An equivalent volume of distilled water was utilized as a control (n=13). Another series of experiments employed retinoic acid (RA: 1 μg) (Oikawa T. et al., Cancer Letters, 48: 157-162 (1989)) (n=12), VEGF (1 μg) (Wilting J. et al., Cell Tissue Res., 274: 163-174 (1993)) (n=10) and an equivalent volume of water (n=10) to validate the CAM model employed, and to provide an internal comparison to the potential angiogenic effects of OGF with other known angioactive agents. These methylcellulose solutions were placed on the tips of 3.2 mm diameter Teflon rods and allowed to dry at room temperature for 1 hour under a vacuum hood. The resultant dried disks were removed from the Teflon rods and placed on the outer third of the CAM. Only one disk was placed on each CAM. The eggs were returned to the incubator for an additional 48 hours of growth prior to examination by image analysis (Mazo J. E. et al., FASEB J., 13: A527 (1999)).

Immunocytochemistry

In order to examine for the presence of OGF and its receptor (OGFr), two eight-day old CAMs were prepared for immunocytochemistry. The CAM and its vessels were dissected from the embryo, placed on a glass slide and frozen by immersion in isopentane cooled by dry ice. The frozen tissue was sectioned (10 μm) and collected on gelatin-coated slides. Sections were fixed in 95% ethanol and acetone, rinsed in Sorenson's phosphate buffer (SPB), and blocked with SPB and 3% normal goat serum for 15 minutes. Immunocytochemistry was performed according to previously published procedures (Zagon I. S. et al., Brain Research, 630: 295-302 (1993); Zagon I. S. et al., J. Invest. Dermal., 196: 490-497 (1996)). In brief, polyclonal immunoglobulin G antibodies to OGF (anti-[Met⁵]-enkephalin, CO-172) and OGFr (AO-440) were diluted 1:100 in SPB with 1% normal goat serum and 0.1% Triton X-100. Tissues were incubated with the primary antibodies in a humidified chamber at 4° C. for 20 hr, washed, and incubated with rhodamine-conjugated goat anti-rabbit IgG (1:100, Cappel Laboratories) for 45 min. Control tissues were sections of CAM stained with antibodies preabsorbed for 24 hr with an excess of the appropriate antigen ([Met⁵]-enkephalin or the receptor binding protein). Matching frozen sections were stained with hematoxylin and eosin for structure identification.

Image Analysis

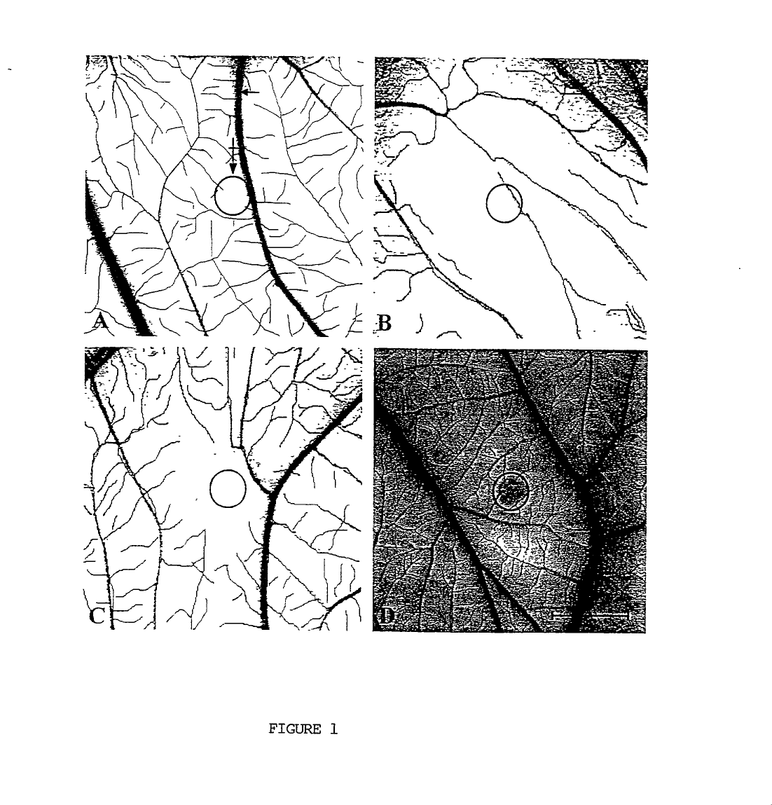

Image analysis was preformed as described previously (Mazo J. E. et al., FASEB J, 13: A527 (1999)). The location of the disk was located visually on the CAM. An identifying mark was placed in the center of the disk and 2 ml of a 10% intralipid solution was injected into the CAM obscuring the underlying yolk vessels and providing an opaque white background for the superficial vessels. The CAM was imaged on a three-chip digital camera (DKC 500 Sony, Tokyo, Japan), digitized, and exported to a personal computer (FIG. 1). A 100 mm² region was defined around the disk in which blood vessel quantitation was performed (Optimas 6.0, Mediacybernetics, Bothell, Wash.). Calibration was achieved by imaging a ruler placed adjacent to the embryo preparation.

All arteries and veins in this designated region centered on the applied disk were analyzed. From the digitized image, an overlying mask was created and all blood vessels were traced within the region in an unblinded manner by two different individuals. The total number of blood vessels were counted and their individual lengths added to obtain total blood vessel length.

For the immunocytochemical specimens, microscopic images were obtained with an Olympus BH-2 microscope equipped for fluorescence microscopy and digitized, as described above. Fluorescence intensity, are, and number of particles was quantified through software imaging analysis (Scion Image Rel 3b; Scion Corp., Frederick, Md.).

Statistics

Data were exported to Microsoft Excel (Excel 97, Microsoft, Redmond, Wash.). All values were expressed as the mean±standard error of the mean. Statistical analysis was performed using the SigmaStat 1.0 statistical software package (Jandel, San Rafael, Calif.). The Mann-Whitney rank sum test or the Student t test was used to compare continuous data. Either the Fischer exact test or χ² test were used for dichotomous data. A p value of less than 0.05 indicated statistical significance.

### RESULTS

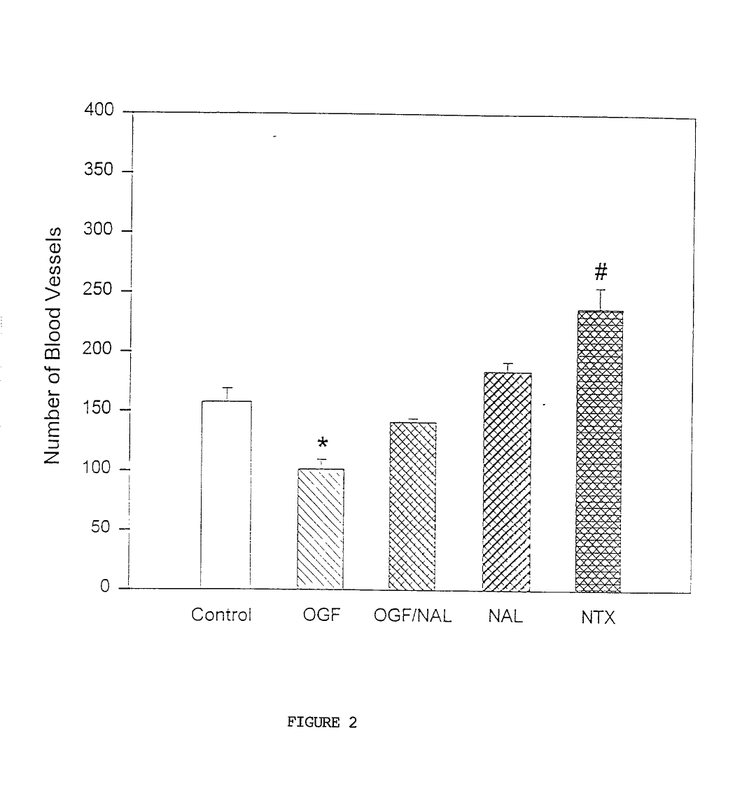

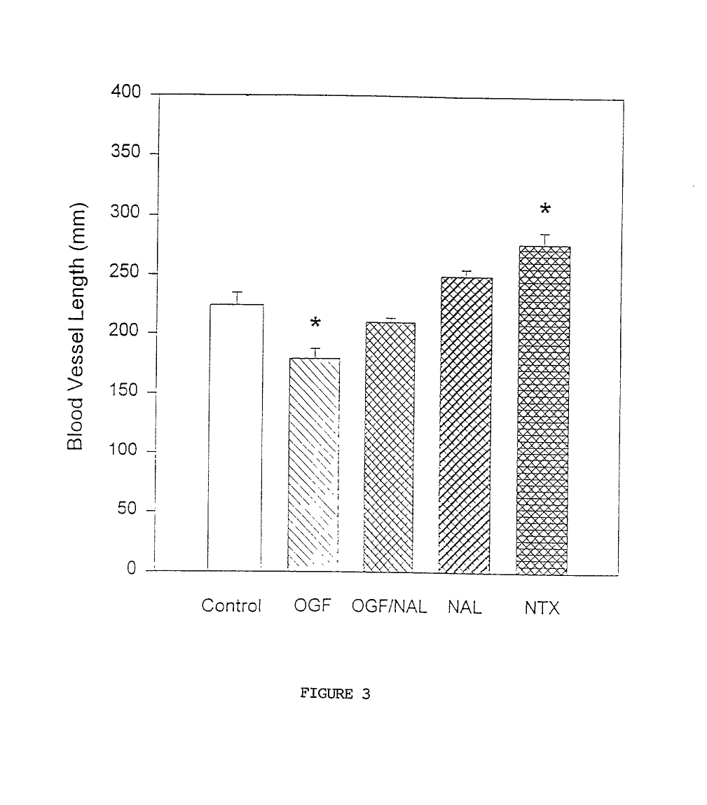

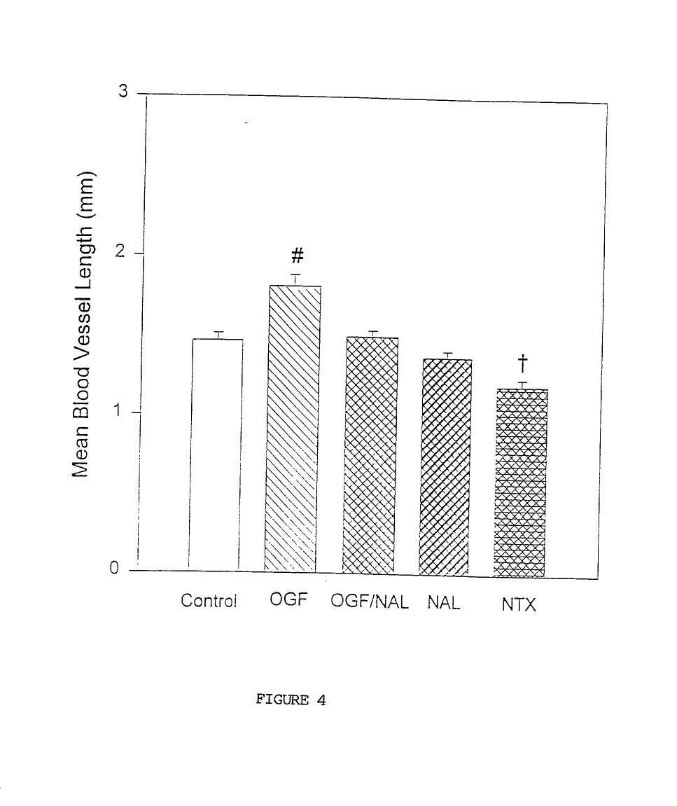

Neither OGF nor its inhibitors appeared to be toxic, as all embryos survived the additional 48 hours after placement of drug onto the CAM. In comparison to controls, application of disks impregnated with the endogenous opioid peptide OGF had a marked inhibitory effect on angiogenesis (FIG. 1C). OGF significantly decreased the number of blood vessels by 35% in comparison to controls (Table I, FIG. 2). Moreover, the OGF group demonstrated a 20% decrease in total blood vessel length in contrast to control CAMs (Table I, FIG. 3). Mean blood vessel length in the OGF group, however, increased significantly from control values (23%) because of a disproportionate decrease in total blood vessel number (35%) (Table I, FIG. 4). These results indicate that OGF is an inhibitory substance associated in vivo with angiogenesis.

To investigate whether the inhibitory effects of OGF on angiogenesis were mediated at the level of the opioid receptor, NAL, a short-acting opioid antagonist was added simultaneously with OGF to the CAM. The concomitant administration of OGF and NAL completely neutralized the inhibitory effect of OGF on angiogenesis as reflected by the number of blood vessels (FIG. 2), total blood vessel length (FIG. 3), and mean blood vessel length (FIG. 4). NAL alone had no effect on vessel growth in comparison to control values. The number and length of blood vessels in the CAMs treated with NAL were significantly greater than the OGF group (FIGS. 2-4) (p<0.0001). These results indicate that the effects of OGF on angiogenesis is receptor mediated.

TABLE I
Effects of Angiogenic Factors on Blood Vessel Growth

| | | | Blood Vessel | |
|---|---|---|---|---|
| Treatment | | Number | Length (mm) | Mean Length (mm) |
| Control | (n = 13) | 157 ± 14 | 224 ± 12 | 1.47 ± 0.06 |
| Opioid Growth | (n = 10) | 102 ± 11* | 179 ± 12* | 1.81 ± 0.13^{#} |
| Factor | | | | |
| Retinoic Acid | (n = 12) | 79 ± 10^{§} | 153 ± 11^{§} | 2.07 ± 0.13^{§} |
| VEGF | (n = 10) | 358 ± 16^{§} | 280 ± 7^{§} | 0.79 ± 0.02^{§} |

Mean ± SEM; Significantly different from controls: *p < 0.005, ^{#}p < 0.001, ^{§}p < 0.0001.

To determine whether angiogenesis reflected the constitutive expression of opioid peptides, the CAM was subjected to the long-acting opioid receptor antagonist Naltrexone (or “NTX”) (FIG. 1D). Preparations subjected to NTX had a 51% increase in the number of blood vessels (FIG. 2), and a 24% increase in total blood vessel length (FIG. 3), relative to control specimens. Mean blood vessel length in the NTX group exhibited a marked decrease in comparison to the control group. These results indicate that NTX, through a persistent blockade of opioid-receptor interaction, inhibited endogenous OGF activity which in turn caused a markedly stimulated blood vessel development.

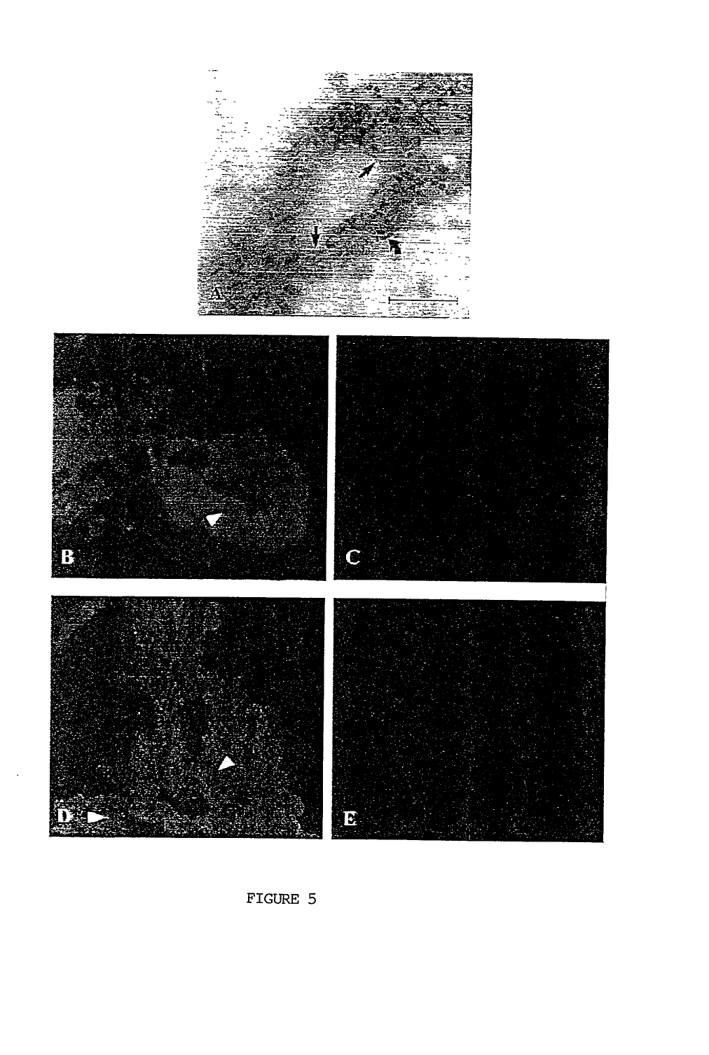

Immunocytochemical analysis demonstrated the presence of both OGF and its receptor in the endothelial cells lining the lumen of the developing vessels and throughout the surrounding mesenchymal cells of the vessel wall (FIG. 5). The peptide and its receptor were localized to the cytoplasm of the cells, and did not appear to be in the cell nuclei. The cumulative area of immunofluorescence was 4,475 μm² for the OGF peptide and 8,280 μm² for the OGFr in a 0.011 mm² region of interest centered on the vessel lumen, compared to no detectable fluorescence (0.0 μm²) for each of their respective controls.

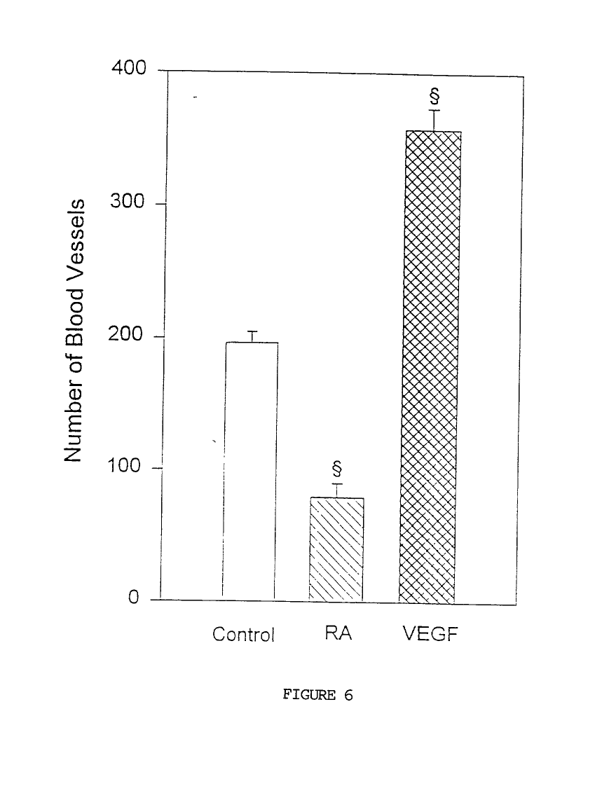

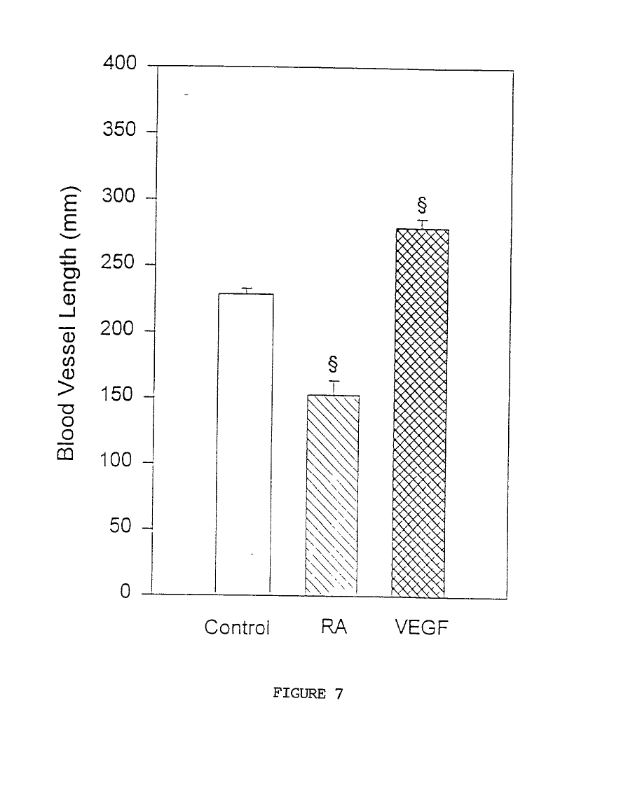

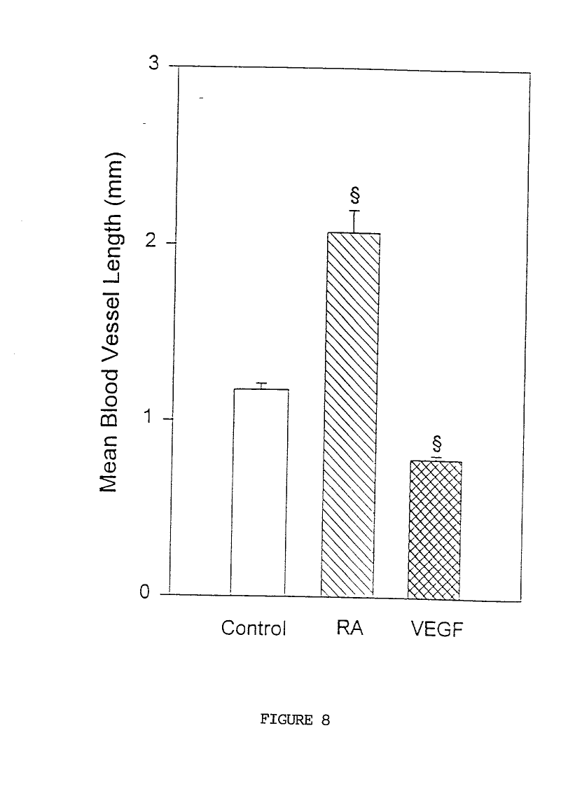

Retinoic acid, a known inhibitor of angiogenesis, (Oikawa T. et al., Cancer Letters, 48: 157-162 (1989); Igniter D. et al., Lab. Invest., 59: 44-51 (1988); Lignin M. W. et al., Lab. Invest., 74(2): 476-483 (1996)) displayed an inhibitory effect on blood vessel growth at the dosage utilized (FIG. 1B, FIGS. 6-8). RA treated CAMs had a 60% decrease in the number of blood vessels (FIG. 6) and a 33% decrease in the total blood vessel length (FIG. 7) compared to the control group (FIGS. 6-7). Mean blood vessel length of the RA treated group, however, increased by 75% relative to the control group (FIG. 8). No significant differences were found between the OGF treated and retinoic acid groups with regard to blood vessel number, vessel length, or mean vessel length.

VEGF, a known stimulant of angiogenesis, (Wilting J. et al., Cell Tissue Res., 274: 163-174 (1993)) displayed an excitatory effect on blood vessel growth (FIGS. 6-8). VEGF increased total vessel length by 22% (FIG. 6), vessel number by 83% (FIG. 7), with an associated decrease in mean blood vessel length of 33% (FIG. 8), in comparison to control specimens. Total vessel length was not different as compared to the NTX treated group but the total number of blood vessels with VEGF was greater (p<0.001) and the mean vessel length was shorter (p<0.0001).

### EXAMPLE 2

The CAM assay described above was utilized. After 3 days of incubation, fertilized chick embryos were explanted and 3.2 mm methylcellulose disk containing a compound was placed onto the CAM surface. After 2 days of growth, the CAM arteries and veins were identified and imaged with a digital camera. Arteries and veins were identified based on the presence of vessel pulsation (arteries), color, and direction of blood flow within the vessels. The main artery was oriented vertically with flow originating inferiorly. A white 10% intralipid solution was injected into the CAM to enhance visualization. Total number and length of veins and arteries were counted and measured. The results are summarized in Table II.

As can be seen from Table II, naltrexone markedly increased both total number and length of all blood vessels in comparison to controls. The mean length of blood vessels decreased in these treated CAMs suggesting the induction of new vessel growth. Naltrexone increased both vein and arterial angiogenesis, maintaining artery/vein ratios for vessel number and length. OGF decreased total number and length of blood vessels in the CAM; however, OGF had a disproportionately greater inhibitory effect on arterial angiogenesis as reflected in decreased artery/vein ratios for vessel number and length.

TABLE II
Differential Effects of Angiogenic Mediators on CAM Preparations

| | | Number of Vessels | | | Total Vessel Length (mm) | | |
|---|---|---|---|---|---|---|---|
| Disk | | Artery | Vein | A/V Ratio | Artery | Vein | A/V Ratio |
| Control | (n = 30) | 119 ± 5 | 69 ± 2 | 2.0 ± 0.1 | 137 ± 3 | 95 ± 3 | 1.6 ± 0.0 |
| VEGF | | | | | | | |
| 0.2 μg | (n = 10) | 83 ± 5* | 75 ± 11 | 1.3 ± 0.1* | 117 ± 3* | 109 ± 14 | 1.2 ± 0.1* |
| 1.0 μg | (n = 14) | 248 ± 13^{#} | 120 ± 7^{#} | 2.2 ± 0.2 | 173 ± 8^{#} | 117 ± 5^{#} | 1.5 ± 0.2 |
| Naltrexone | | | | | | | |
| 0.1 μg | (n = 10) | 80 ± 6 | 83 ± 4 | 1.0 ± 0.1 | 112 ± 6 | 118 ± 6 | 1.0 ± 0.1 |
| 5.0 μg | (n = 11) | 203 ± 7* | 97 ± 4* | 2.1 ± 0.1 | 174 ± 4* | 105 ± 2 | 1.7 ± 0.0 |
| OGF | | | | | | | |
| 1.0 μg | (n = 9) | 53 ± 5* | 56 ± 8 | 1.1 ± 0.2 | 84 ± 5^{§} | 81 ± 11 | 1.2 ± 0.2 |
| 5.0 μg | (n = 9) | 79 ± 4* | 51 ± 2* | 1.6 ± 0.1* | 107 ± 4* | 82 ± 3 | 1.3 ± 0.1 |
| Retinoic acid | (n = 9) | 60 ± 5* | 39 ± 3* | 1.5 ± 0.1* | 83 ± 5* | 66 ± 4* | 1.3 ± 0.1* |

Data represents mean ± SEM.
Significantly different from group controls at *p < 0.05 using ANOVA with post-hoc testing.
Significantly different from controls at ^{§}p < 0.005, ^{#}p < 0.0001 using a two-tailed t-test.

What is claimed is:

1. A method of inhibiting angiogenesis in a tissue or organ of a subject in need thereof, which comprises administering a therapeutically effective amount of OGF to the subject.

2. A method of inhibiting angiogenesis in a tissue or organ of a subject in need thereof, which comprises administering a therapeutically effective amount of naltrexone sufficient to cause intermittent blockade of OGF receptors.

3. The method of claim 1 or 2, wherein said tissue or organ comprises hemangiomas, ocular neovascularization, tumors, metastatic tumors, venous malformations, varicose veins, arterio-venous malformations, vascular tumors, intimal hyperplasia, and/or atherosclerotic arterial stenosis or occlusions.

4. The method of claim 1 or 2, wherein the administration is by injection via an oral, ophthalmic, nasal, topical, transdermal, parenteral, intracranial, intracerebral, intraspinal, intravaginal, intrauterine, or rectal route.

5. The method of claim 1 or 2, wherein the administration is by surgical implantation proximate to a preselected tissue or organ site in need of inhibition of angiogenesis.

6. The method of claim 1, wherein OGF is administered intravenously in the amount of about 10 mg to about 30 mg per day to a human subject.

7. The method of claim 1, wherein OGF is administered subcutaneously in the amount of 1 mg/ml to about 5 mg/ml per day to a human subject.

8. The method of claim 1, wherein OGF is provided in a pharmaceutically acceptable carrier.

9. The method of claim 8, wherein said carrier is oil, water, saline solution, gel, lipid, liposome, or a porous matrix material.

10. The method of claim 9, wherein said carrier is capable of a controlled release of OGF.

11. The method of claim 2, wherein naltrexone is administered in the amount of about 1 mg to about 5 mg per day.

12. A method of enhancing angiogenesis in a tissue or organ of a subject in need thereof, which comprises administering a therapeutically effective amount of an OGF antagonist to the subject.

13. The method of claim 12, wherein said tissue or organ is a damaged tissue or organ, or a transplant tissue or organ.

14. The method of claim 13, wherein said tissue is cardiac tissue, nervous tissue, skin, tissue of gastrointestinal tract, or tissue of urogenital tract, epithelium, muscular tissue, bone, cartilage, pulmonary issue, eye, oral cavity, glands, or connective tissue.

15. The method of claim 12, wherein said OGF antagonist is selected from naloxone, naltrexone, an antibody against OGF, an antibody against the OGF receptor, or an antisense molecule of the OGF-receptor-encoding gene.

16. The method of claim 15, wherein said antagonist is naltrexone or naloxone, administered in an amount sufficient to persistently block OGF receptors.

17. The method of claim 16, wherein naltrexone is administered in the amount of about 10 mg to about 30 mg per day.

18. The method of claim 16, wherein naloxone is administered in the amount of about 1 mg to about 5 mg per dose for multiple doses per day.

19. The method of claim 12, wherein said OGF antagonist is provided in a pharmaceutically acceptable carrier.

20. The method of claim 19, wherein said carrier is oil, water, saline solution, gel, lipid, liposome, or a porous matrix material.

21. The method of claim 20, wherein said carrier is capable of a controlled release of said OGF antagonist.

22. The method of claim 12, wherein said OGF antagonist is administered by injection via an oral, ophthalmic, nasal, topical, transdermal, parenteral, intracranial, intracerebral, intraspinal, intravaginal, intrauterine, or rectal route.

23. The method of claim 22, wherein said OGF antagonist is administered by surgical implantation proximate to a preselected tissue or organ site in need of angiogenesis.

24. A pharmaceutical composition, comprising a pharmaceutically acceptable carrier and an angiogenesis-inhibiting amount of OGF.

25. A pharmaceutical composition, comprising a pharmaceutically acceptable carrier and an amount of naltrexone sufficient to cause intermittent blockade of OGF receptors and inhibit angiogenesis.

26. A pharmaceutical composition, comprising a pharmaceutically acceptable carrier and an angiogenesis-enhancing amount of an OGF antagonist.

27. The pharmaceutical composition of claim 23, wherein said OGF antagonist is selected from naloxone, naltrexone, an antibody against OGF, an antibody against the OGF receptor, or an antisense molecule of the OGF-receptor-encoding gene.

28. The pharmaceutical composition according to any one of claims 24-26, wherein said carrier is oil, water, saline solution, gel, lipid, liposome, or a porous matrix material.

29. The method of claim 28, wherein said carrier is capable of controlled release.
